(19) United States
(12) Patent Application Publication
(10) Pub. No.: US 2018/0078463 A1
(43) Pub. Date: Mar. 22, 2018

(54) THERMOMETER-EQUIPPED PACIFIER CAPABLE OF SELF-CHARGING USING PIEZOELECTRIC ELEMENT

(71) Applicant: Industry-Academic Cooperation Foundation, Yonam Institute of Technology, Jinju-si (KR)

(72) Inventor: DaeSung JUNG, Jinju-si (KR)

(73) Assignee: Industry-Academic Cooperation Foundation, Yonam Institute of Technology, Jinju-si (KR)

(21) Appl. No.: 15/293,621

(22) Filed: Oct. 14, 2016

(30) Foreign Application Priority Data
Sep. 19, 2016 (KR) 10-2016-0119211

(51) Int. Cl. A61J 17/00; A61B 5/01; A61B 5/00; A61M 21/02

(52) U.S. Cl. CPC A61J 17/003; A61J 17/001; A61B 5/01; A61M 2021/0027; A61M 21/02; A61B 5/0022; A61B 2560/0219; A61B 5/742

(57) ABSTRACT

Disclosed herein is a thermometer-equipped pacifier capable of self-charging using a piezoelectric element. The pacifier includes a nipple part, a case, a temperature sensor unit, a piezoelectric element unit, a charging unit, a display unit, and a control unit. The nipple part is made of a flexible elastic material, and has an internal space. The case is coupled to the lower portion of the nipple part. The temperature sensor unit is disposed inside the nipple part, and detects body temperature. The piezoelectric element unit is disposed inside the nipple part, and generates electricity in response to external pressure. The charging unit is contained in the case, and charges and stores the electricity. The display unit is attached onto the case, and displays the body temperature. The control unit is contained in the case, and performs control so that power can operate the temperature sensor unit and the display unit.

### BACKGROUND

### 1. Technical Field

The present invention relates generally to a pacifier that an infant plays with as a toy or an infant uses to fall sleep, and more particularly to a thermometer-equipped pacifier capable of self-charging using a piezoelectric element, in which a digital thermometer is contained in a pacifier, and thus the body temperature of an infant can be checked as desired and the pacifier can perform self-charging.

### 2. Description of the Related Art

An infant product called a pacifier (American English), a dummy (United Kingdom, other Commonwealth countries and Ireland), a binky or soother (Canadian English), and a teether (Philippine English) is an essential item that is used by almost all infants.

FIG. 1 is a photo showing a typical pacifier.

In general, a pacifier includes a nipple-shaped nipple part “a” made of a transparent and elastic material so that an infant can have the nipple part “a” in his or her mouth and can bite or suck it, a guard plate “b” configured to prevent the pacifier from being fully inserted into the mouth, and a case “c,” as shown in the photo.

Since there are many cases where infants always have such pacifiers in their mouths, inventions that are configured such that a temperature sensor is disposed in a pacifier and thus a the body temperature of an infant can be checked as desired have been proposed.

FIG. 2 is an exploded perspective view showing a conventional thermometer-equipped pacifier.

As shown in FIG. 2, the conventional thermometer-equipped pacifier includes: a nipple-shaped nipple part 10 made of a flexible elastic material, and configured to have an internal space; a guard plate 12 securely combined with the lower end portion of the nipple part 10 to be integrated with the nipple part 10, and configured to have a hole communicating with the space of the nipple part 10; a digital thermometer 4 configured to be combinable with the guard plate 12 and to include a temperature sensing element 18 inserted deeply into the nipple part 10; and a melody generator 6 configured to be combinable with the guard plate 12 and to issue one or more stored lullabies using an IC circuit.

The temperature sensor is inserted into the nipple part, and thus body temperature is indirectly measured and is displayed using a digital method, thereby providing the advantage of enabling an observer to be immediately aware of the body temperature of an infant. Furthermore, the conventional thermometer-equipped pacifier is configured to issue a melody, such as a lullaby, thereby providing the advantage of enabling an infant to easily fall asleep while having the pacifier in his or her mouth.

However, the conventional thermometer-equipped pacifier is inconvenient in that a battery must be replaced when the battery is discharged because a separate battery is contained in the pacifier and supplies power. The digital thermometer accelerates discharge speed because it is always in a turned on state. Furthermore, while a melody is being issued, the voltage consumed by the speaker is considerably high.

[Preceding Technical Documents]

[Patent Documents]

1) Korean Utility Model Registration No. 20-0214472 published on Dec. 11, 2000 and entitled “Multipurpose Pacifier with Separable Thermometer”

2) Japanese Utility Model Registration No. 3161231 published on Jul. 29, 2010 and entitled “Nipple Type Thermometer”

3) U.S. Pat. No. 5,108,423 published on Apr. 28, 1992 and entitled “Nipple Assembly with Alarm Buzzer and Body Temperature Indicator”

### SUMMARY

Accordingly, the present invention is intended to overcome the above-described problems of the conventional art, and an object of the present invention is to provide a thermometer-equipped pacifier capable of self-charging using a piezoelectric element, in which a piezoelectric element is provided inside a pacifier, and thus electricity is generated and charged by using external pressure generated when an infant bites or sucks the pacifier, with the result that the supply of external power is not required.

In order to achieve the above object, there is provided a thermometer-equipped pacifier capable of self-charging using a piezoelectric element, including: a nipple-shaped nipple part made of a flexible elastic material, and configured to have an internal space; a case coupled to the lower portion of the nipple part; a temperature sensor unit disposed inside the nipple part, and configured to detect body temperature in the mouth of an infant; a piezoelectric element unit disposed inside the nipple part, and configured to generate electricity through deformation thereof attributable to external pressure; a charging unit contained in the case, and configured to charge and store the electricity generated by the piezoelectric element unit; a display unit attached onto the outside of the case, and configured to display the temperature detected by the temperature sensor unit; and a control unit contained in the case in a circuit board form, and configured to perform control so that power supplied by the charging unit can operate the temperature sensor unit and the display unit.

The nipple part may be filled with an elastic filler material so that the external pressure can be directly transferred to the piezoelectric element.

The case may further contain a sound source unit configured to store and play back source sounds and a speaker configured to output the source sounds played back by the sound source unit, and the control unit may perform control so that power can be supplied to the sound source unit and the speaker.

The case may further contain a wireless transmission unit configured to receive the body temperature of the infant from the control unit and transmit body temperature data to an external mobile device, and the control unit may perform control so that power can be supplied to the wireless transmission unit.

The case may further contain a charging socket connected to the charging unit and configured to enable charging through connection with an external power source.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a photo showing a typical pacifier;

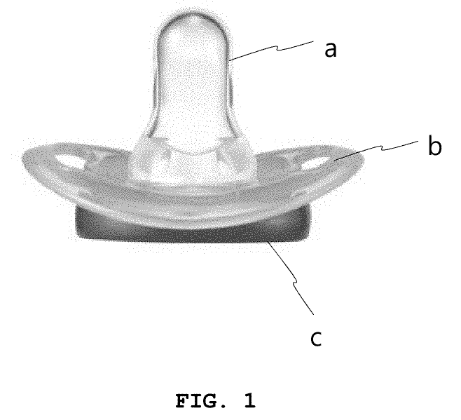

FIG. 2 is an exploded perspective view showing a conventional thermometer-equipped pacifier;

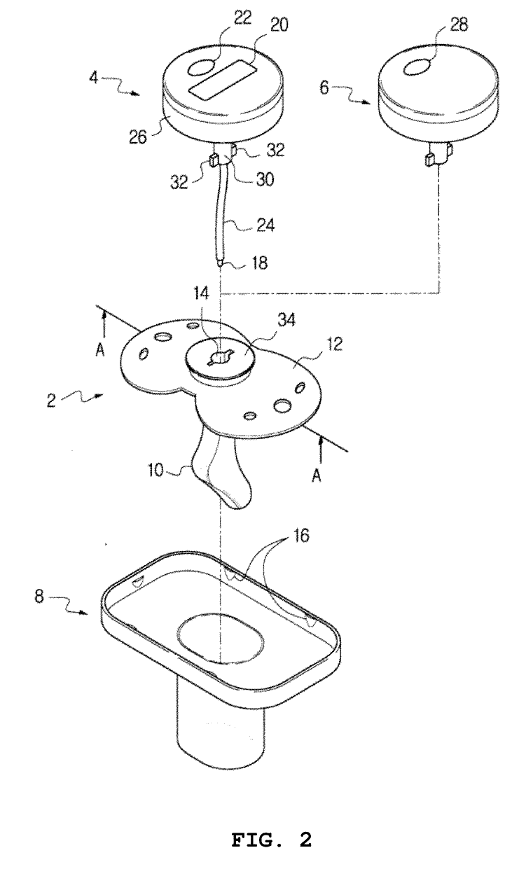

FIG. 3 is a perspective view showing the overall appearance of a thermometer-equipped pacifier capable of self-charging using a piezoelectric element according to an embodiment of the present invention;

FIG. 4 is a sectional view of the thermometer-equipped pacifier shown in FIG. 3;

FIG. 5 is a diagram showing the overall circuit configuration of a thermometer-equipped pacifier capable of self-charging using a piezoelectric element according to the present invention; and

FIG. 6 is a sectional view showing a thermometer-equipped pacifier capable of self-charging using a piezoelectric element according to another embodiment of the present invention.

### DETAILED DESCRIPTION

Although the present invention will be described in conjunction with embodiments of the present invention and with reference to the accompanying drawings, the embodiments of the present invention are intended to help to easily understand the present invention and the scope of the present invention is not limited to the embodiments of the present invention.

In the following description, when it is determined that detailed descriptions of known components or functions may unnecessarily make the gist of the present invention obscure, they will be omitted.

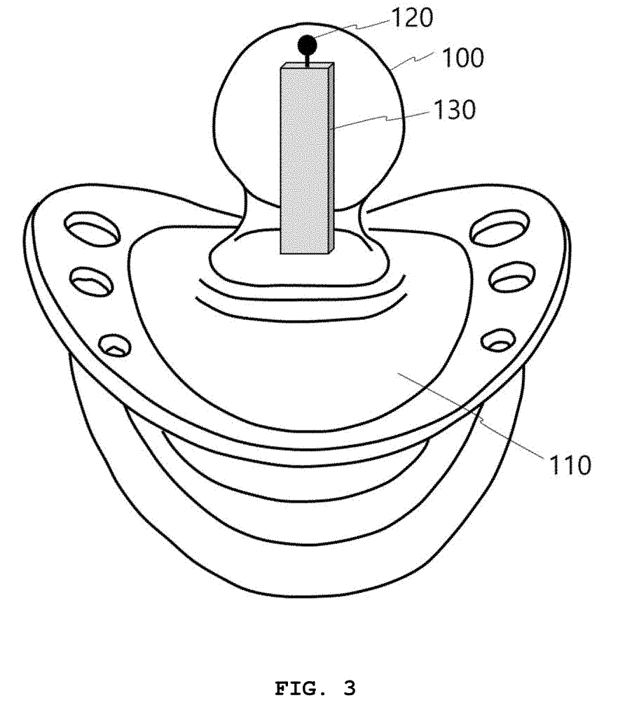

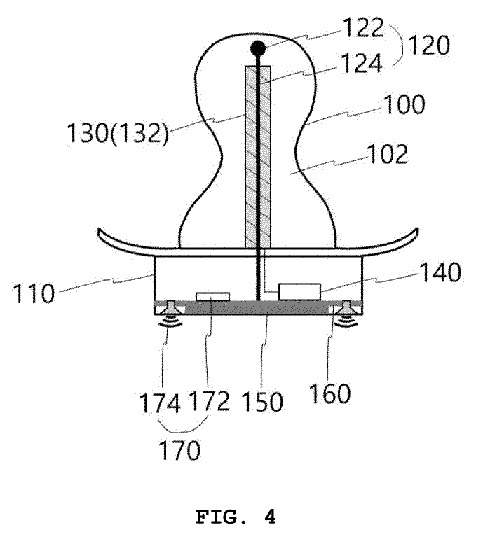

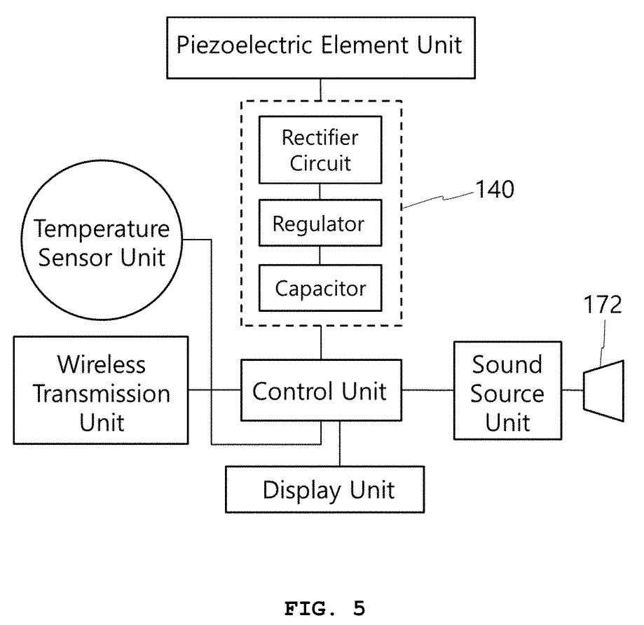

FIG. 3 is a perspective view showing the overall appearance of a thermometer-equipped pacifier capable of self-charging using a piezoelectric element according to an embodiment of the present invention, FIG. 4 is a sectional view of the thermometer-equipped pacifier shown in FIG. 3, and FIG. 5 is a diagram showing the overall circuit configuration of a thermometer-equipped pacifier capable of self-charging using a piezoelectric element according to the present invention.

Referring to FIGS. 3 to 5, a thermometer-equipped pacifier capable of self-charging using a piezoelectric element according to the present invention may be configured to include a nipple part 100, a case 110, a temperature sensor unit 120, a piezoelectric element unit 130, a charging unit 140, a display unit 150, and a control unit 160.

First, the nipple part 100 is made of flexible elastic material, and has an internal space. Although it is generally preferred that the nipple part 100 has a nipple shape so that an infant can have the nipple part 100 in his or her mouth, the shape of the nipple part 100 is not limited to any specific shape.

The nipple part 100 may be made of a well-known material, such as elastic rubber, silicone, or synthetic resin harmless to human bodies.

Furthermore, a transparent material, an opaque material, or a translucent material may be used.

Next, the case 110 is combined with the nipple part 100. The case 110 has an internal space, and may accommodate the charging unit 140, the display unit 150, and the control unit 160 therein.

The case 110 is generally made of a plastic material. Although not shown in the drawings, a hole that communicates with the nipple part 100 is formed through the top of the case 110. Through this hole, the temperature sensor unit 120 and the piezoelectric element unit 130 accommodated in the nipple part 100 are electrically connected to the control unit 160 disposed in the case 110.

Next, the temperature sensor unit 120 is disposed to be accommodated inside the nipple part 100. The temperature sensor unit 120 functions to detect the body temperature of an infant.

The temperature sensor unit 120 may be a well-known digital temperature sensor, and may be of a contact type. The contact type sensor may detect temperature when contact with the nipple part 100 is maintained. Since an infant has the nipple part 100 in his or her mouth continuously, sufficient time required for the temperature sensor unit 120 to detect body temperature is ensured.

Meanwhile, a variety of non-contact type temperature sensors recently commercialized may be used. The non-contact type sensors can detect temperature without contact, and thus can accurately detect temperature.

Meanwhile, the temperature sensor unit 120 measures body temperature, and thus a wide temperature measurement range is not required.

In the temperature sensor unit 120, a temperature sensor 122 is disposed to be contained in the upper end portion of the nipple part 100 in order to desirably detect temperature. An electric wire 124 configured to transfer signals may be extended and connected to the inside of the case 110.

Next, the piezoelectric element unit 130 that corresponds to a core technical feature of the present invention will be described.

The piezoelectric element unit 130 includes a piezoelectric element configured to generate electricity in response to external pressure, and is also disposed inside the nipple part 100. The reason for this is to enable the external pressure, generated when an infant bites or sucks the nipple part 100, to be directly transferred to the piezoelectric element 132.

The external pressure that is transferred to the piezoelectric element 132 may include various types of force, such as compressive force, bending force, twisting force, etc. Accordingly, the piezoelectric element 132 may be perpendicularly disposed, as in the temperature sensor unit 120 shown in the drawings. The piezoelectric element 132 may be contained in the nipple part 100 in the form in which the piezoelectric element 132 is combined with the electric wire 124 of the temperature sensor unit 120. However, this disposition is merely an example. Another type of disposition that enables external pressure to be desirably transferred may be employed.

Furthermore, the piezoelectric element unit 130 may be of a flexible type, and may have various shapes, such as a rod shape, a plate shape, etc. Accordingly, a piezoelectric polymer element, such as a polyvinylidene fluoride (PVDF) element, may be used.

In this case, although the nipple part 100 may have an empty space therein, the nipple part 100 may be configured to be filled with an elastic filler material 102 so that there is no empty space therein, with the result that the external pressure can be directly transferred to the piezoelectric element 132. Furthermore, the filler material functions to allow heat transfer to be more effectively performed when the above-described contact type temperature sensor is contained, thereby enabling the effective detection of body temperature.

In this case, the filler material 102 may be identical or similar to the material of the nipple part 100. That is, the filler material 102 may be silicone, rubber, or the like.

Next, the charging unit 140 is disposed inside the case 110, and is configured to receive, charge and store the electrical energy generated by the piezoelectric element unit 130.

As shown in FIG. 5, the charging unit 140 may include a rectifier circuit, a regulator, and a capacitor.

The rectifier circuit rectifies AC current generated by the piezoelectric element unit 130. The DC current obtained through the rectification is regulated by the regulator to have a predetermined voltage. Thereafter, the regulated DC current is transferred to the capacitor, and may be subjected to charging.

In this case, the rectifier circuit may have an arrangement in which four diodes are arranged in a bridge form, and the regulator may include a converter to convert voltage.

Next, the display unit 150 is configured to display the temperature, detected by the temperature sensor unit 120, on a screen so that the temperature can be recognized from the outside.

The display unit 150 is attached onto the outside of the case 110, and may employ a general LCD digital screen connected to the control unit 160 to be described later.

Next, the control unit 160 is disposed inside the case 110 in the form of a printed circuit board (PCB). The control unit 160 is connected to the temperature sensor unit 120, the piezoelectric element unit 130, the charging unit 140, and the display unit 150, and controls them.

The control unit 160 receives the detected temperature from the temperature sensor unit 120 in an electrical form, processes and converts the received temperature data, and displays the processed and converted temperature data via the display unit 150 in a numerical form. Furthermore, the control unit 160 performs control so that the electricity generated by the piezoelectric element unit 130 can be charged into the charging unit 140, and also performs control so that the charged power can be continuously supplied to the temperature sensor unit 120 and the display unit 150.

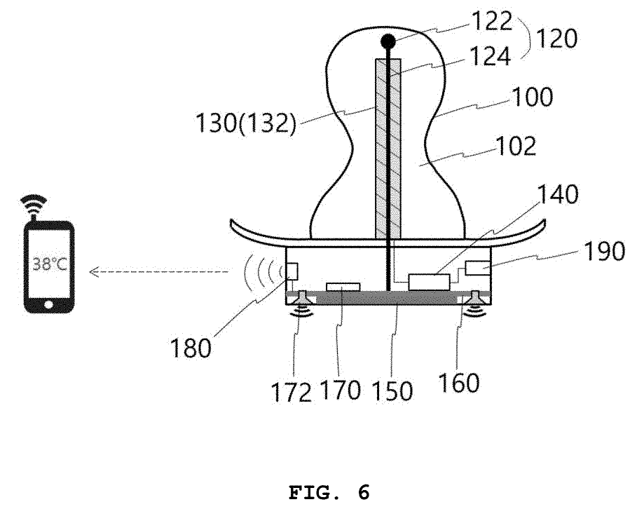

FIG. 6 is a sectional view showing a thermometer-equipped pacifier capable of self-charging using a piezoelectric element according to another embodiment of the present invention. The thermometer-equipped pacifier capable of self-charging using a piezoelectric element according to the other embodiment of the present invention will be described with reference to FIG. 6.

According to the present embodiment of the present invention, a sound source unit 170 configured to store and play back source sounds in order to play back melodies to an infant is disposed inside a case 110.

The sound source unit 170 may include memory configured to store source sounds, and a playback circuit configured to play back the source sounds stored in the memory.

Furthermore, a speaker 172 may be further included in order to output the source sounds, played back by the sound source unit 170, to the outside. The speaker 172 may be disposed on the outside of the case 110, as shown in FIG. 6. A pair of speakers 172 may be provided on both sides of the display unit 150.

In this case, the control unit 160 performs control so that power can be supplied from a charging unit 140 to the memory and the playback circuit, and also performs control so that music is output from the sound source unit 170 via the speaker 172. It will be apparent that a switch configured to selectively turn on and off the power of the speaker 172 and a volume control may be further included.

Alternatively, in the present embodiment of the present invention, a wireless transmission unit 180 may be further included in the case 110, may receive the body temperature of an infant from a control unit 160, and may transmit the received body temperature to an external mobile device.

That is, as shown in FIG. 6, the wireless transmission unit 180 connected to the control unit 160 receives power, and the temperature data measured by the temperature sensor unit 120 is displayed via a display unit 150 and, simultaneously, transmitted to the outside via the wireless transmission unit 180 continuously or regularly.

The transmission to a mobile device may be performed using wireless communication methods, such as LTE, Bluetooth, WiFi and ZigBee methods, etc., and thus the wireless transmission unit 180 may employ a device, such as a Bluetooth module, a WiFi module, a ZigBee module, etc. Since these communication methods correspond to well-known technologies, detailed descriptions thereof are omitted.

Accordingly, a parent can check and deal with the body temperature of an infant, transmitted by the wireless transmission unit 180, via a mobile device as desired. A configuration may be made such that an alarm is issued when the body temperature of an infant is equal to or higher than a predetermined threshold body temperature.

Additionally, in an embodiment of the present invention, a separate charging socket 190 may be further included in order to prepare for a case where power is fully discharged from the capacitor of the charging unit 140.

The charging socket 190 is disposed inside the case 110, and is configured to perform charging through internal connection with the battery and external connection with an external power source.

The operation of the present invention will be described below.

Since an infant has the nipple part 100 in his or her mouth and bites or sucks the nipple part 100, the nipple part 100 is exposed to various types of external pressure, such as compressive pressure, bending pressure, and twisting pressure.

As a result, the piezoelectric element unit 130 disposed inside the nipple part 100 is compressed, bent or twisted by external pressure, electricity is generated, and the generated electricity is charged into the capacitor through the rectifier circuit and the regulator in a current form.

The control unit 160 performs control so that the power charged into the charging unit 140 can be supplied to the temperature sensor unit 120, the display unit 150, the sound source unit 170, the speaker 172, and the wireless transmission unit 180, and also performs control so that the body temperature detected by the temperature sensor unit 120 can be converted into digital data and displayed on the display unit 150.

Furthermore, the source sounds stored in the sound source unit 170 are played back using various methods and output to the outside via the speaker 172, thereby helping an infant sleep deeply.

Moreover, the wireless transmission unit 180 transmits the body temperature data of an infant to an external mobile device, thereby helping a parent check the body temperature of the infant as desired.

According to the above-described present invention, the nipple part of the pacifier that is used as a toy by an infant is held in his or her mouth and is continuously subjected to external pressure, and thus self-charging is performed by the piezoelectric element, with the result that the pacifier can be semi-permanently used as a thermometer.

Furthermore, an infant can sleep deeply by means of the sound source unit, and a parent can check and deal with the body temperature of an infant via a mobile device at a remote location by means of the wireless transmission unit.

Although the present invention has been described in conjunction with the specific embodiments of the present invention and with reference to the accompanying drawings, it will be apparent to those having ordinary knowledge in the art to which the present invention pertains that various modifications and alterations can be made within the scope of the present invention based on the above detailed description.

What is claimed is:

1. A thermometer-equipped pacifier capable of self-charging using a piezoelectric element, comprising:
a nipple-shaped nipple part made of a flexible elastic material, and configured to have an internal space;
a case coupled to a lower portion of the nipple part;
a temperature sensor unit disposed inside the nipple part, and configured to detect body temperature in a mouth of an infant;
a piezoelectric element unit disposed inside the nipple part, and configured to generate electricity through deformation thereof attributable to external pressure;
a charging unit contained in the case, and configured to charge and store the electricity generated by the piezoelectric element unit;
a display unit attached onto an outside of the case, and configured to display the temperature detected by the temperature sensor unit; and
a control unit contained in the case in a circuit board form, and configured to perform control so that power supplied by the charging unit can operate the temperature sensor unit and the display unit.

2. The thermometer-equipped pacifier of claim 1, wherein the nipple part is filled with an elastic filler material so that the external pressure can be directly transferred to the piezoelectric element.

3. The thermometer-equipped pacifier of claim 1, wherein the case further contains a sound source unit configured to store and play back source sounds and a speaker configured to output the source sounds played back by the sound source unit, and the control unit performs control so that power can be supplied to the sound source unit and the speaker.

4. The thermometer-equipped pacifier of claim 1, wherein the case further contains a wireless transmission unit configured to receive the body temperature of the infant from the control unit and transmit body temperature data to an external mobile device, and the control unit performs control so that power can be supplied to the wireless transmission unit.

5. The thermometer-equipped pacifier of claim 1, wherein the case further contains a charging socket connected to the charging unit and configured to enable charging through connection with an external power source.
